(19) United States
(12) Patent Application Publication
(10) Pub. No.: US 2004/0096528 A1
(43) Pub. Date: May 20, 2004

(54) Compositions and methods for preserving personal care products

(76) Inventors: Daniel A. Miser, Provo, UT (US); Chong Wang, Draper, UT (US); Jamie Anderson, Pleasant Grove, UT (US); Matthew R. Lewis, American Fork, UT (US)

Correspondence Address: WORKMAN NYDEGGER (F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 10/606,486

(22) Filed: Jun. 26, 2003

Related U.S. Application Data
(60) Provisional application No. 60/391,757, filed on Jun. 26, 2002.

(51) Int. Cl. A61K 35/78

(52) U.S. Cl. 424/773

(57) ABSTRACT

Personal care product preservation systems and methods comprise the use of wasabi and a co-preservative, such as pentylene glycol or a mixture of undecylenoyl glycine and capryloyl glycine to preserve a personal care product. The wasabi provides a natural preservative effect that inhibits the growth of microbes that can otherwise flourish in personal care products. The personal care products can be in the form of emulsions, gels, serums, solutions, and other vehicles suitable for use with personal care products, such as toners, lotions, creams, and the like. Personal care product preservation systems and methods may also comprise the mixture of undecylenoyl glycine and capryloyl glycine to preserve a personal care product.

### CROSS-REFERENCE TO RELATED APPLICATIONS

This application claims priority under 35 U.S.C. § 119(e) to U.S. provisional application Serial No. 60/391,757, filed Jun. 26, 2002. For purposes of disclosure, the foregoing application is incorporated herein in its entirety.

### BACKGROUND OF THE INVENTION

1. The Field of the Invention

The present invention relates generally to all-natural compositions and methods for preserving personal care products. More particularly, the invention relates to the use of wasabi and it derivatives as a preservative for personal care products.

2. The Relevant Technology

Water-based personal care products, such as lotions and toners, provide an hospitable environment for microbial growth. Microbes can be introduced to personal care products, for example, during manufacture, storage, and/or consumer use. How quickly the microbes grow and render the product unsuitable for use is a function of various factors, including the susceptibility of individual products to microbial growth, the presence or absence of preservatives, and the storage condition of the personal care product after the microbial contamination first occurs.

As a result, personal care products have defined shelf lives to ensure a product's quality. These shelf lives typically include two critical dates: a shelf date, which is an absolute limit on a product's life, regardless of whether the packaging is opened by a consumer; and an expiration date, which may be shorter than the shelf date, depending upon when the user opens the packaging. For example, a product may have a two year expiration date after a product is opened by a consumer, subject to an absolute shelf life of three years.

Obviously, the longer a product's shelf life the greater its value, both to a manufacturer concerned about product quality and maintaining inventories and to a consumer concerned about how quickly a product must be consumed. There is therefore a constant need to develop effective methods to deter microbial growth and thereby extend product shelf lives.

Typically, artificial preservatives are used to extend product shelf lives. There is a growing consumer awareness and fear, however, that artificial preservatives may contain damaging chemicals whose effect on users or the environment may be harmful, either individually over time or in a cumulative effect with other chemicals. There is therefore also a need for products that preclude microbial growth without the use of artificial preservatives. This presents a difficult challenge to providing “organic” and “all natural” products that nevertheless have suitable shelf lives.

One somewhat effective approach is to carefully package a product in sanitary conditions and then hermetically seal the product to prevent the introduction of microbes. This only prevents product degradation prior to consumer use, however, as an un-preserved product may quickly degrade after an initial consumer use, such as after microbial-laden fingers contact the product. Alternatively, intricate product dispensing systems, such as pumps, may provide effective methods to delay contamination but cannot slow microbial growth once contamination occurs.

U.S. Pat. No. 6,361,812 to Ekanayake et al. (“Ekanayake”) discloses a preservative system for use in e.g., foods, beverages, health care products, personal care products, herbicidal products, and containment devices, comprising one or more isothiocyanates and an artificial preservative selected from sorbate preservatives, benzoate preservatives, and mixtures thereof. The isothiocyanate compounds may be manufactured synthetically or isolated from natural plant matter.

In view of the foregoing, there is a continuing need for more organic and natural systems and compositions to preserve personal care products.

### SUMMARY OF THE INVENTION

The present invention relates to personal care product preservation systems and methods that use wasabi japonica (“wasabi”) and, optionally, a co-preservative to preserve a personal care product. Wasabi has been found to provide a natural preservative effect that inhibits the growth of microbes that can otherwise flourish in aqueous based personal care products. The personal care products can be in any desired form, including emulsions, gels, serums, solutions, and other vehicles suitable for use with personal care products, such as toners, lotions, creams, cleansers, refiners, and the like. Unlike artificial preservatives, including synthesized compounds and single compounds isolated from natural products, wasabi is a true natural product.

Accordingly, in one personal care product preservation system according to the invention, a personal care product is blended with a preservative composition comprising wasabi and pentylene glycol. One preferred form of wasabi is wasabia japonica root extract. Suitable concentrations of the preservative may vary according to the personal care product formulation being preserved. For example, the concentration of water is often an important variable because microbial growth generally follows water concentrations. The pentylene glycol provides a co-preservative effect.

According to another embodiment of the invention, a personal care product preservation system comprises a personal care product and a preservative composition comprising wasabia japonica, undecylenoyl glycine, and capryloyl glycine. The undecylenoyl glycine and capryloyl glycine are collectively believed to provide a co-preservative effect. The use of other organic co-preservatives, including variations and derivatives of the foregoing, is also contemplated by the invention.

Similarly, a method according to the invention for inhibiting microbial growth in a personal care product comprises mixing a personal care product, a wasabia root japonica extract, and at least one additional co-preservative selected from the group consisting of pentylene glycol, undecylenoyl glycine, and capryloyl glycine.

These and other advantages and features of the present invention will become more fully apparent from the following description and appended claims, or may be by the practice of the invention as set forth hereinafter.

### DETAILED DESCRIPTION OF THE INVENTION

I. Introduction.

Personal care product preservation systems and methods comprise the use of wasabia japonica (“wasabi”) as a preservative to inhibit microbial growth in personal care products. The wasabi provides a natural preservative without the use of undesirable artificial preservatives. When used in selected concentrations and pH ranges and with appropriate co-preservatives, the wasabi can be an effective preservative.

The optional co-preservative, for example pentylene glycol or a mixture of undecylenoyl glycine and capryloyl glycine, helps preserve the personal care product and ensure that only safe levels of wasabi need to be used. The use of a co-preservative is important because certain personal care products may require high preservative concentrations to prevent all microbial growth. Because high concentrations of wasabi may cause skin irritation, the present invention uses co-preservatives to allow effective microbial growth control at low wasabi concentrations.

According to another embodiment of the invention undecylenoyl glycine and capryloyl glycine can be used without wasabi to inhibit microbial growth in personal care products. These two compositions have been found to have a synergistic effect that can provide an effective approach to inhibiting microbial growth.

The personal care products can be in any desired form including emulsions, gels, serums, solutions, and other vehicles. Such vehicles are commonly used in consumer products such as toners, lotions, creams, refiners, cleansers, and the like.

In addition to the surprising effectiveness of wasabi as a preservative for personal care products, wasabi has the advantage of being an all-natural product. In contrast, conventional preservatives typically comprise synthetic chemical compounds, and are therefore disfavored by many consumers for their potential health and environmental risks. The use of wasabi as an alternative preservative to synthetic chemical compositions provides not only reduced health and environmental risks, to the extent that such exist, but also satisfies the consuming public's demand for natural products.

Additional advantages of the present invention will become readily apparent to those skilled in the art from the following detailed description, wherein currently preferred embodiments of the invention are shown and described in the disclosure. As will be realized, the invention is capable of other and different embodiments, and its several details are capable of modifications in various obvious respects, all without departing from the invention. Accordingly, the description is to be regarded as illustrative in nature, and not as restrictive.

In the following description, numerous specific details are set forth in order to provide a thorough understanding of the present invention. It will be obvious, however, to one skilled in the art that the present invention may be practiced without these specific details. In other instances, well known aspects of personal care products have not been described in particular detail in order to avoid unnecessarily obscuring the present invention.

II. Compositions

A. Preservatives

According to the invention, the primary preservative is wasabia japonica (“wasabi”), a member of the cruciferae family. The wasabi plant grows about knee high, is semi aquatic, and produces a thickened stem in a similar fashion to a small brussel sprout. As the stem grows the lower leaves fall off. This stem has a very pungent smell and flavor when made into a paste, which is commonly used as a condiment for sushi. Wasabi can be produced both as a ground grown or water grown plant, although water-grown plants generally produce a higher quality product than the ground grown plants.

Like its relatives in the cabbage family, wasabi owes its pungency to isothiocyanates. In addition, two glucosinolates have been identified in the root: sinigrin, which is also the characteristic aroma compound of black mustard and horseradish, and glucocochlearin, found in trace quantities. These tasteless compounds are enzymatically hydrolyzed to the pungent “mustard oils” allyl isothiocyanate and sec-butyl isothiocyanate, respectively. Further trace components are 6-methylthiohexyl isothiocyanate, 7-methylthioheptyl isothiocyanate and 8-methylthioocytl isothiocyanate. These compounds are generally classified as omega-methylthioalphyl isothiocyanates.

One preferred form to include wasabi in the formulations of the present invention is wasabia japonica root extract, which can be obtained from Active Concepts, LLC in S. Plainfield, N.J. The wasabia japonica root is extracted using a method proprietary to Active Concepts that involves the following general methods: fermentation of the dry plant matter in water; initial extraction using a potassium phosphate aqueous solution; and separation of all molecular fractions below 5,000 MW using a tangential flow filtration method to provide the commercial material. Other methods of manufacturing wasabi extract include enzymatic extraction or sequentially macerating plant matter in water or other solvents and then percolating the plant matter in a solvent extraction process.

Despite the effectiveness of wasabi as a preservative, it has been determined that it is most effective when utilized in combination with at least one co-preservative. The use of a co-preservative is advantageous because it enables the use of lower wasabi concentrations than might otherwise be required to control microbial growth. When high wasabi concentrations are required to preserve a product, skin irritation with clinical manifestations, e.g. erythema, and/or sub-clinical manifestations, e.g. itching, is possible. The use of co-preservatives thus allows the selection of an optimized wasabi concentration that balances the desired preservative action with minimized skin irritation.

One such co-preservative is pentylene glycol (C₅H₁₂O₂), an organic compound that is commercially available from Dragoco, Inc. in Totowa, N.J. Although pentylene glycol has been used in combination with chemical preservatives, such as parabens, its synergistic co-preservative effect with natural substances has not been heretofore established.

Another co-preservative is the combination of capryloyl glycine and undecylenoyl glycine. Glycines are amino acids commonly found in plants, and capryloyl glycine (C₁₀H₁₉NO₃) is the acylation product of glycine with caprylic acid chloride. Undecylenoyl Glycine is the acylation product of glycine with n-undecylenoyl chloride. Both capryloyl glycine and undecylenoyl glycine are commercially available from SEPPIC, in Paris, France under the trade names Lipacide C8G and Lipacide UG, respectively.

Other suitable natural co-preservatives, including variations and derivatives of the foregoing, may also be used and are compatible with the present invention.

Suitable concentrations of wasabi to preserve personal care products ranges from a low of about 0.1% to a high of about 10% by weight of the overall composition. Nevertheless, wasabi ranges substantially above about 5% may result in skin irritation, and therefore a preferred range is from about 3% to about 6% by weight of the composition. In the case of low water content formulations, which generally has a lower susceptibility to microbial growth or where wasabi may be used to boost the effect of other preservatives or co-preservatives, a lower range of wasabi is also effective, preferably from about 0.1% to about 1% by weight of the composition.

Suitable concentration ranges for co-preservatives will vary depending on the selected co-preservative, the concentration of wasabi, and the product being preserved.

B. Vehicles

The systems of the invention can employ various vehicles, or mediums, depending on the particular form of personal care product. Common vehicles include solutions, emulsions, gels, and serums, each of which can be used to provide the desirable form for consumer products, such as toners, lotions, creams, cleansers, refiners, and the like. These varied vehicles enable the present invention to be used in the wide range of personal care products that are commercially available.

Gels and serums are similar vehicles, the main difference being that gels tend to have a high viscosity whereas serums have a low viscosity. Oil and water emulsions can be either oil-in-water emulsions or water-in-oil emulsions, either one being suitable for use with consumer care products. However, the ratio of oil-to-water can greatly affect the concentration of preservatives required. This is because microbes tend to flourish or die, depending on the amount of water present in a formulation. Thus, an emulsion with 1% oil and 99% water generally requires higher concentrations of wasabi preservative then an emulsion with 99% oil and 1% water. For example, an emulsion of 50% to 99% in the oil phase and 1% to 50% in the water phase may require wasabi in a concentration of from about 0.1% to about 5% by weight of the composition, whereas an emulsion of 1% to 50% in the oil phase and 50% to 100% in the water phase may require wasabi in a concentration of from about 0.1% to about 10% by weight of the composition.

Typical components of the herein discussed vehicles (solutions, gels, serums, and emulsions) vary depending on the particular formulation, but may include water, emulsifiers, co-emulsifiers, thickeners, surfactants, gellants, humectants, moisturizers, botanicals, solubilizers, acidulents, neutralizers, emollients, organic sunscreens, inorganic powders, astringents, chelating agents, solvents, detergents, essential oils, and colorants.

In addition, it has been determined that the vehicles of the herein disclosed systems will preferably have a pH value in a range from about 2 to about 8, more preferably from about 3 to about 7, and most preferably from about 5 to about 6.

Methods of preserving personal care products involves the application of the foregoing teachings regarding the use of wasabi preservative compositions to inhibit microbial growth in personal care products. Hence, the methods involve mixing the appropriate amounts of wasabi and the desired co-preservative into the chosen vehicle of the personal care product, such as solutions, gel, serum, or emulsion. Other packaging techniques (sealed containers, sanitary pumps) may also assist in inhibiting microbial growth.

The following examples are given to illustrate the present invention, and are not intended to limit the scope of the invention.

### EXAMPLES

Ten consumer product formulations were prepared according to the foregoing teachings to incorporate the use of wasabia japonica root extract as a preservative. These formulations are presented in the following Examples 1-10. The concentrations are in weight percent.

### Example 1

### Day Energy Serum

| COMPONENT | PERCENT |
|---|---|
| Water | 76.6270 |
| Wasabia Japonica Extract | 5.0000 |
| Glycerine | 2.5000 |
| Dimethicone | 2.5000 |
| Pentylene Glycol | 2.0000 |
| Methyl Gluceth-20 | 1.5000 |
| Polysorbate-20 | 0.5000 |
| Xanthan Gum | 0.3000 |
| Carbomer | 0.1500 |
| Sodium Hydroxide | 0.0520 |
| Tetrasodium EDTA | 0.0500 |
| Others | 8.8210 |
| Total | 100.0000 |

### Example 2

### Night Recovery Serum

| COMPONENT | PERCENT |
|---|---|
| Purified Water | 65.3190 |
| Glycerine | 7.0000 |
| Cyclomethicone/Dimethicone/Phenyl Trimethicone | 7.0000 |
| Butylene Glycol | 5.0000 |
| Wasabia Japonica Root Extract | 5.0000 |
| Dimethicone Copolyol | 2.0000 |
| Cyclomethicone | 2.0000 |
| Pentylene Glycol | 2.0000 |
| Polysorbate 20 | 0.8500 |
| Xanthan Gum | 0.2500 |
| Carbomer | 0.1500 |
| Tetrasodium EDTA | 0.0500 |
| Sodium Hydroxide | 0.0400 |
| Others | 3.3410 |
| Total | 100.0000 |

### Example 3

### Hydrating Refiner

| COMPONENT | PERCENT |
|---|---|
| Purified Water | 89.3240 |
| Wasabia Japonica Root Extract | 3.5000 |
| Polysorbate-20 | 0.9000 |
| Pentylene Glycol | 0.8900 |
| Benzopheneone-4 | 0.0500 |
| Tetrasodium EDTA | 0.0450 |
| Others | 5.2910 |
| Total | 100.0000 |

### Example 4

### Mattefying Refiner

| COMPONENT | PERCENT |
|---|---|
| Purified Water | 89.5350 |
| Wasabia Japonica Root Extract | 3.5000 |
| Polysorbate-20 | 1.5000 |
| Pentylene Glycol | 1.5000 |
| PEG-40 Hydrogenated Castor Oil/Tridecth-9 | .6000 |
| Benxophenone-4 | .0500 |
| Tetrasodium EDTA | .0500 |
| Others | 3.2650 |
| Total | 100.0000 |

### Example 5

### Day Veil Creme

| COMPONENT | PERCENT |
|---|---|
| Purified Water | 45.8590 |
| Cyclopentasiloxane Dimethicone/Vinyl | 12.0000 |
| Dimethicone Crosspolymer | |
| Ethylhexyl Methoxycinnamate | 6.0000 |
| Glyceryl Stearate | 6.0000 |
| PEG-40 Stearate | 3.5000 |
| Butylene Glycol | 2.0000 |
| Pentylene Glycol | 1.0000 |
| Stearic Acid | 2.5000 |
| Glycerin | 2.0000 |
| Cetyl Octanote | 2.0000 |
| Cyclomethicone | 1.5000 |
| Dimethicone | 1.5000 |
| Titanium Dioxide | 1.0000 |
| Sodium Carbomer | 0.3500 |
| Wasabia Japonica Root Extract | 0.3000 |
| Chlorphenesin | 0.2000 |
| Methylparaben | 0.2500 |
| Disodium EDTA | 0.0500 |
| Others | 11.9910 |
| Total | 100.0000 |

### Example 6

### Night Nourishing Creme

| COMPONENT | PERCENT |
|---|---|
| Purified Water | 61.5530 |
| Cyclopentasiloxane Dimethicone/Vinyl | 14.0000 |
| Dimethicone Crosspolymer | |
| Pentylene Glycol | 3.0000 |
| Cetyl Octanoate | 2.0000 |
| Dimethicone | 2.0000 |
| Ethylhexyl Methoxycinnamate | 1.5000 |
| Sodium Carbomer | 0.5000 |
| Acrylates/C10-30 Alkyl Acrylate Crosspolymer | 0.5000 |
| Wasabia Japonica Root Extract | 0.3000 |
| PEG-75 | 0.2500 |
| Chlorphenesin | 0.2000 |
| Methylparaben | 0.2500 |
| Tetrasodium EDTA | 0.0500 |
| Others | 13.8970 |
| Total | 100.0000 |

### Example 7

### Night Nourishing Lotion

| COMPONENT | PERCENT |
|---|---|
| Purified Water | 65.5530 |
| Isohexadecane | 6.5000 |
| Wasabia Japonica Root Extract | 5.0000 |
| Pentylene Glycol | 3.0000 |
| Cyclopentasiloxane | 3.0000 |
| Hydrogenated Polyisobutene | 2.5000 |
| Ethyihexyl Methoxycinnamate | 0.5000 |
| Laureth-7/Polyacrylamide/C13-14 Isoparaffin/Water | 0.5000 |
| Acrylates/C10-30 Alkyl Acrylate Crosspolymer | 0.4000 |
| Sodium Hydroxide | 0.1000 |
| Xanthan Gum | 0.1500 |
| Tetrasodium EDTA | 0.0500 |
| Others | 12.7470 |
| Total | 100.0000 |

### Example 8

### Day Veil Lotion

| COMPONENT | PERCENT |
|---|---|
| Purified Water | 58.5390 |
| Cyclopentasiloxane Dimethicone/Vinyl | 10.0000 |
| Dimethicone Crosspolymer | |
| Ethylhexyl Methoxycinnamate | 6.0000 |
| Glyceryl Stearate SE | 3.0000 |
| Stearic Acid | 2.5000 |
| Butylene Glycol | 2.0000 |
| Glycerin | 2.0000 |
| Pentylene Glycol | 1.0000 |
| Titanium Dioxide | 1.0000 |
| Wasabia Japonica Root Extract | 0.3000 |
| Carbomer | 0.2500 |
| Chlorphenesin | 0.2000 |
| Methylparaben | 0.1900 |
| Sodium Hydroxide | 0.0400 |
| Others | 12.9810 |
| Total | 100.0000 |

### Example 9

### Creamy Cleanser

| COMPONENT | PERCENT |
|---|---|
| Purified Water | 62.7290 |
| Canola Oil | 8.0000 |
| C12-15 Alkyl Benzoate | 5.0000 |
| Glycerin | 2.6000 |
| Pentylene Glycol | 2.6000 |
| Glyceryl Stearate and PEG-100 Stearate | 2.5000 |
| Stearic Acid | 2.0000 |
| Polysorbate 20 | 2.0000 |
| Capryloyl Glycine | 1.0000 |
| Euphorbia Cerifera (Candelilla) Wax | 1.0000 |
| Cetyl Alcohol | 0.9500 |
| Sodium Hydroxide | 0.5400 |
| Undecylenoyl Glycine | 0.5000 |
| Ethylhexyl Methoxycinnamate | 0.5000 |
| Citric Acid | 0.5000 |
| Xanthan Gum | 0.4000 |
| Wasabia Japonica Root Extract | 0.3000 |
| Carbomer | 0.2000 |
| Sodium Metabisulfite | 0.0500 |
| Tetrasodium EDTA | 0.0500 |
| Others | 6.5810 |
| Total | 100.0000 |

### Example 10

### Foaming Cleanser

| COMPONENT | PERCENT |
|---|---|
| Purified Water | 31.3340 |
| Sodium Lauroyl Oat Aminoacids | 20.0000 |
| PEG-120 Methyl Glucose Dioleate | 5.0000 |
| Cocamidopropyl Betaine | 3.5000 |
| Decyl Glucoside | 3.0000 |
| Pentylene Glycol | 1.0000 |
| Vegetable Glycerin | 1.5000 |
| Capryloyl Glycine | 1.0000 |
| Undecylenoyl Glycine | 0.5000 |
| Sodium Hydroxide | 0.6200 |
| Wasabia Japonica Root Extract | 0.3000 |
| PEG-75 | 0.2500 |
| Polysorbate 20 | 0.2000 |
| Citric Acid | 0.5000 |
| Benzo henone-4 | 0.1000 |
| Tetrasodium EDTA | 0.0500 |
| Others | 31.1460 |
| Total | 100.0000 |

The effectiveness of the above formulations was challenge tested according to USP 24 guidelines. The specified method comprised the basic steps of: (1) sterilizing a sample; (2) inoculating each formula sample with the micro-organisms as specified by the USP 24; (3) analyzing the sample at intervals of 0, 7, 14, 21, and 28 days to quantify the test substance; and (4) validating the counting procedure. Challenge testing under USP 24 guidelines prescribes that for a Category 1B product (e.g. Creamy Cleanser and Day Energy Serum), there be no less than a 2 log reduction from day 1 to day 14 and no increase in colony count from day 14 to day 28. Each of the above formulations satisfied the respective challenge test.

As indicated in each of Examples 11 and 12, these criteria were satisfied as to Creamy Cleanser and Day Energy Serum with each of the test substances staphylococcus aureus ATCC 6538, escherichia coli ATCC 8739, pseudomonas aeuruganosa ATCC 9027, candida albicans ATCC 10231, and aspergillus niger ATCC 16404.

### Example 11

### Preservative Challenge for Creamy Cleanser

| Test Substance | Initial | Day 7 | Day 14 | Day 21 | Day 28 |
|---|---|---|---|---|---|
| Staphylococcus aureus | 3.2e5 | 700 | <10 | <10 | <10 |
| ATCC 6538 | | | | | |
| Escherichia coli ATCC | 3.2e5 | 4200 | <10 | <10 | <10 |
| 8739 | | | | | |
| Pseudomonas | 6.2e5 | <10 | <10 | <10 | <10 |
| acuruganosa ATCC 9027 | | | | | |
| Candida albicans ATCC | 6.6e5 | 300 | 5000 | <10 | <10 |
| 10231 | | | | | |
| Aspergillus niger ATCC | 2.9e5 | <30,000 | 7000 | <10 | <10 |
| 16404 | | | | | |

### Example 12

### Preservative Challenge for Day Energy Serum

| Test Substance | Initial | Day 7 | Day 14 | Day 21 | Day 28 |
|---|---|---|---|---|---|
| Staphylococcus aureus | 3.2e5 | <10 | <10 | <10 | <10 |
| ATCC 6538 | | | | | |
| Escherichia coli ATCC | 3.2e5 | <10 | <10 | <10 | <10 |
| 8739 | | | | | |
| Pseudomonas aeuruganosa | 6.2e5 | <10 | <10 | <10 | <10 |
| ATCC 9027 | | | | | |
| Candida albicans ATCC | 6.6e5 | <10 | <10 | <10 | <10 |
| 10231 | | | | | |
| Aspergillus niger ATCC | 2.9e5 | 9300 | 400 | <10 | <10 |
| 16404 | | | | | |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A composition of matter comprising:
a personal care product; and
a preservative composition, dispersed within the personal care product, that inhibits growth of microbes within the personal care product, comprising:
wasabia japonica; and
at least one co-preservative selected from the group comprising pentylene glycol, undecylenoyl glycine, and capryloyl glycine.

2. The composition of claim 1, wherein the wasabia japonica comprises wasabia japonica root extract.

3. The composition of claim 1, wherein the wasabia japonica is present in a concentration of about 0.1% to about 10% by weight of the composition.

4. The composition of claim 1, wherein the wasabia japonica is present in a concentration of about 3% to about 6% by weight of the composition.

5. The composition of claim 1, further comprising an additional preservative, wherein the wasabia japonica is present in a concentration of from about 0.1% to about 1% by weight of the composition.

6. The composition of claim 1, wherein the co-preservative is present in a concentration of from about 0.1% to about 10% by weight of the composition.

7. The composition of claim 1, wherein the composition of matter has a pH is a range from about 2 to about 8.

8. The composition of claim 1, wherein the composition of matter has a pH in a range from about 3 to about 7.

9. The composition of claim 1, wherein the composition of matter has a pH in a range from about 5 to about 6.

10. The composition of claim 1, wherein the personal care product comprises a vehicle selected from the group comprising gels, serums, emulsions and solutions.

11. The composition of claim 10, wherein the vehicle comprises an oil-in-water emulsion or a water-in-oil emulsion.

12. The composition of claim 1, wherein the personal care product comprises an emulsion of 50% to 99% in the oil phase and 1% to 50% in the water phase, wherein the wasabi is present in a concentration of from about 0.1% to about 5% by weight of the composition.

13. The composition of claim 1, wherein the composition of matter comprises an emulsion of 1% to 50% in the oil phase and 50% to 100% in the water phase, wherein the wasabi is present in a concentration of from about 0.1% to about 10% by weight of the composition.

14. The composition of claim 1, wherein the personal care product is selected from the group consisting of a lotion, a cleanser, a refiner, a toner, and a cream.

15. A composition of matter comprising:
a personal care product; and
a preservative composition for inhibiting the growth of microbes within the personal care product, comprising:
wasabia japonica; and
at least one co-preservative comprising at least one of (i) pentylene glycol or (ii) undecylenoyl glycine and capryloyl glycine.

16. A composition of matter comprising:
a personal care product; and
a preservative mixture for inhibiting the growth of microbes within the personal care product, comprising:
undecylenoyl glycine, and
capryloyl glycine.

17. The composition of claim 16, wherein the preservative mixture is present in a concentration of from about 0.1% to about 10% by weight of the composition.

18. The composition of claim 16, wherein the composition of matter has a pH in a range from about 3 to about 7.

19. The composition of claim 16, wherein the composition of matter has a pH in a range from about 5 to about 6.

20. The composition of claim 16, wherein the personal care product comprises a vehicle selected from the group comprising gels, serums, emulsions and solutions.

21. The composition of claim 20, wherein the vehicle comprises an oil-in-water emulsion or a water-in-oil emulsion.

22. The composition of claim 16, wherein the vehicle comprises an emulsion of 50% to 99% in the oil phase and 1% to 50% in the water phase.

23. The composition of claim 16, wherein the vehicle comprises an emulsion of 1% to 50% in the oil phase and 50% to 100% in the water phase.

24. The composition of claim 16, wherein the personal care product is selected from the group comprising a lotion, a cleanser, a refiner, a toner, and a cream.

25. A method for inhibiting microbial growth in a personal care product, comprising:
mixing together a personal care product, wasabia japonica, and at least one co-preservative selected from the group comprising pentylene glycol, undecylenoyl glycine, and capryloyl glycine.

26. The method of claim 25, wherein the personal care product is selected from the group consisting of a lotion, a cleanser, a refiner, a toner, and a cream.
